(19) United States
(12) Patent Application Publication
(10) Pub. No.: US 2021/0316184 A1
(43) Pub. Date: Oct. 14, 2021

(54) METHOD FOR MONITORING EXERCISE SESSION WITH MULTIPLE SCHEMES

(71) Applicant: bOMDIC, Inc., New Taipei (TW)

(72) Inventors: HSIN-FU KUO, New Taipei (TW); HAN-TSUNG PAN, New Taipei (TW); CHIEN-YI CHIU, New Taipei (TW); TAI-YU HUANG, New Taipei (TW)

(21) Appl. No.: 16/843,853

(22) Filed: Apr. 8, 2020

(51) Int. Cl. A63B 24/00; G06F 17/15

(52) U.S. Cl. CPC A63B 24/0062; A63B 2230/04; A63B 2024/0068; G06F 17/15

(57) ABSTRACT

The present invention discloses a method for monitoring an exercise session of a user. The exercise session comprises a first portion and a second portion following the first portion. Provide an exercise record of a previous exercise of the user, wherein the exercise record comprises a first exercise correspondence associated with a first fitness condition of the user. Execute a first exercise scheme to monitor the first portion of the exercise session based on the first exercise correspondence. Execute a second exercise scheme to monitor the second portion of the exercise session based on a comparison of a second fitness condition of the user derived from an exercise data in the first portion of the exercise session and the first fitness condition of the user in the exercise record of the previous exercise.

### BACKGROUND OF THE INVENTION

### 1. Field of the Invention

The present invention relates to a method for monitoring an exercise session, and more particularly to a method for monitoring an exercise session with multiple schemes.

### 2. Description of Related Art

It is common and effective to use a monitoring device to help people to improve their health and exercise. Therefore, it is very important that the monitoring device must precisely provide the personalized exercise data for the user.

In the conventional exercise-monitoring method, a real-time exercise is monitored only by the exercise correspondence associated with the fitness condition in the long-term exercise record; however, it is hard to monitor this exercise by the exercise correspondence associated with the real-time fitness condition. The fitness condition of the user may vary with sick, diet, sleep, stress or environment (e.g., weather). If the above conditions happen, the real-time fitness condition of the user may be worse and then there is much difference between the real-time fitness condition and the fitness condition in the long-term exercise record. Because of the difference of the fitness condition, the conventional exercise-monitoring method may be not effectively applied in a real-time exercise. Furthermore, if the conventional exercise-monitoring method associated with the high fitness condition in the long-term exercise record is applied in a real-time exercise which the user of the low fitness condition takes, the user will be overloaded or get hurt.

Accordingly, the present invention proposes a method for monitoring an exercise session with multiple schemes to overcome the above-mentioned disadvantages.

### SUMMARY OF THE INVENTION

In the early portion of the exercise session, the present invention precisely and quickly judges if the real-time fitness condition is consistent with the fitness condition (e.g., the fitness condition of the exercise record of the previous exercise) used in the exercise scheme monitoring the early portion of the exercise session to further determine if a modified exercise scheme is needed to monitor the later portion of the exercise session. Preferably, the present invention chooses a reasonable parameter (or a combination of the multiple parameters) representing the fitness condition to precisely and quickly if the real-time fitness condition is consistent with the fitness condition used in the exercise scheme monitoring the early portion of the exercise session. If the real-time fitness condition is not consistent with the fitness condition used in the exercise scheme monitoring the early portion of the exercise session, execute a modified exercise scheme to monitor the later portion of the exercise session by the fitness condition used in the modified exercise scheme being closer to the real-time fitness condition of the user than that used in the unmodified exercise scheme. If the real-time fitness condition of the user is worse, the modified exercise scheme is executed to monitor the later portion of the exercise session such that the user can be prevented from being overloaded or getting hurt.

By the algorithm implemented in the computer of the present invention, the computer of the present invention performs operations described in claims or the following descriptions to precisely monitor the latter portion of the exercise session.

In one embodiment, the present invention discloses a method for monitoring an exercise session of a user, wherein the exercise session comprises a first portion and a second portion following the first portion. The method comprises steps of: providing, by a memory unit, an exercise record of a previous exercise of the user, wherein the exercise record comprises a first exercise correspondence associated with a first fitness condition of the user; executing, by a processing unit, a first exercise scheme to monitor the first portion of the exercise session based on the first exercise correspondence; and executing, by the processing unit, a second exercise scheme to monitor the second portion of the exercise session based on a comparison of a second fitness condition of the user derived from an exercise data in the first portion of the exercise session and the first fitness condition of the user in the exercise record of the previous exercise.

In one embodiment, the present invention discloses a method for monitoring an exercise session of a user, wherein the exercise session comprises a first portion and a second portion following the first portion. The method comprises steps of: providing, by a memory unit, an exercise record of a previous exercise of the user, wherein the exercise record comprises a first exercise correspondence associated with a first fitness condition of the user; executing, by a processing unit, a first exercise scheme to monitor the first portion of the exercise session based on the first exercise correspondence; and executing, by the processing unit, a second exercise scheme to monitor the second portion of the exercise session based on a comparison of a second fitness condition of the user derived from an exercise data in the first portion of the exercise session and the first fitness condition of the user in the exercise record of the previous exercise; wherein a parameter representing the first fitness condition has a first value and the parameter representing the second fitness condition has a second value, wherein the comparison of the second fitness condition and the first fitness condition is the comparison of the second value of the parameter and the first value of the parameter.

The detailed technology and above preferred embodiments implemented for the present invention are described in the following paragraphs accompanying the appended drawings for people skilled in the art to well appreciate the features of the claimed invention.

### BRIEF DESCRIPTION OF THE DRAWINGS

The foregoing aspects and many of the accompanying advantages of this invention will become more readily appreciated as the same becomes better understood by reference to the following detailed description when taken in conjunction with the accompanying drawings, wherein:

FIG. 1 illustrates a method for executing the multiple schemes to monitor an exercise session of a user in the present invention;

FIG. 2 illustrates a schematic block diagram of an exemplary apparatus in the present invention;

FIG. 3 illustrates a method for monitoring an exercise session of a user in the present invention;

FIG. 4 illustrates a first exercise correspondence associated with the first fitness condition of the user in one embodiment of the present invention;

FIG. 5 specifically shows that the descending rate of the fitness index 2.5% per minute in the bottom curve is much larger than that 1% per minute in the beginning portion of the top curve when the user runs at the speed of 8 kilometers per hour;

FIGS. 6A and 6B illustrate that execute the second exercise scheme to monitor the second portion of the exercise session in Embodiment 1A of the present invention;

FIGS. 6C and 6D illustrate that execute the second exercise scheme to monitor the second portion of the exercise session in Embodiment 1B of the present invention;

FIG. 7A illustrates that execute the second exercise scheme to monitor the second portion of the exercise session in Embodiment 2A of the present invention;

FIG. 7B illustrates that execute the second exercise scheme to monitor the second portion of the exercise session in Embodiment 2B of the present invention; and

FIG. 7C illustrates that execute the second exercise scheme to monitor the second portion of the exercise session in Embodiment 2C of the present invention.

### DETAILED DESCRIPTION OF THE ILLUSTRATED EMBODIMENTS

The detailed explanation of the present invention is described as following. The described preferred embodiments are presented for purposes of illustrations and description and they are not intended to limit the scope of the present invention.

### Definition of the Terms

Fitness Condition

The fitness condition may be defined by the fitness performance level. The fitness performance level of one user may be different from that of the other user; if two users want to achieve the same training effect, one user of more fitness performance level needs acuter exercise guiding and higher exercise intensity than the other user of less fitness performance level. The fitness performance level may include health-related fitness and sport/skill-related fitness which can be also improved by engaging in physical activities or exercise training. For example, the parameter of the fitness performance level may be VO₂ₘₐₓ or METₘₐₓ (maximum oxygen uptake capacity relative to resting oxygen consumption: equal to VO₂ₘₐₓ/3.5), and VO₂ₘₐₓ is preferred. Generally, a unit of the VO₂ₘₐₓ can be represented in an absolute way, such as oxygen uptake (ml/min), or in a relative way, such as oxygen uptake based on weight (ml/kg/min).

Fitness Index

Fitness index (e.g., stamina) is used to evaluate the amount of energy someone has before exercise or after at least one portions of exercise. Low fitness index represents that body is unrecovered. After at least one portions of exercise, a person needs to reduce the exercise intensity or take a rest to recover the consumed fitness index.

Cardiac Output Power

Cardiac output power may be a body's internal reaction to the external workload (e.g., speed and altitude data from GPS or accelerometer, pedaling power or other kinetic data created by workload causing energy expenditure). Because the heart rate is easily available and it is accurate to depict the exercise response, the heart rate is the most preferable variable to get the internal response. In the present invention, the heart rate is used as one example of the cardiac output power; however, the cardiac output power may comprise other parameters (e.g., HRV, pulse, oxygen consumption or respiration rate) different from the heart rate.

Exercise Intensity

Exercise intensity may refer to how much energy is expended when exercising. Exercise intensity may define how hard the body has to work to overcome a task/exercise. Conventionally, the heart rate may be used as one exercise intensity measurement or used as an indicator of exercise intensity; however, the measured heart rate may not reflect the current exercise intensity, for example, under the condition that the anaerobic threshold is exceeded. In order to exclude internal reaction/response like the heart rate, the exercise intensity in the present invention represents the exercise intensity of the external workload (e.g., speed and altitude data from GPS or accelerometer, pedaling power or other kinetic data created by the external workload resulting in energy expenditure).

Mathematical Correspondence

The mathematical correspondence may describe the correlation between two parameters and provide a sufficient match between two parameters. The mathematical correspondence may be a function describing the dependence of one parameter on the other parameter. The mathematical correspondence can be estimated by a linear regression model, a non-linear regression model, or any other suitable mathematical models. Further, the mathematical correspondence may describe the correlation among three or more parameters and provide a sufficient match among three or more parameters.

Exercise Correspondence

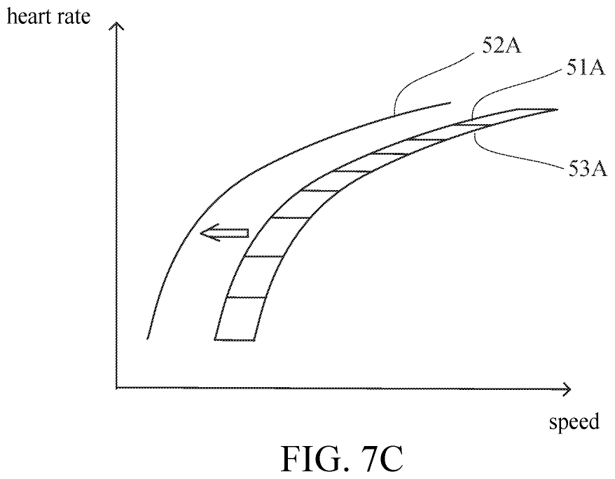

The exercise correspondence may be an above-mentioned mathematical correspondence. The exercise correspondence may be a natural correspondence describing the correlation between two parameters (e.g., exercise-physiological parameter) without a mathematical computation. The natural correspondence may be a function describing the dependence of one parameter on the other parameter. The exercise correspondence may be in the form of a zone; for example, the zone may have a left boundary of the first exercise correspondence 51A and a right boundary of the third exercise correspondence 53A shown in FIG. 7C. Further, the mathematical correspondence may describe the correlation among three or more parameters.

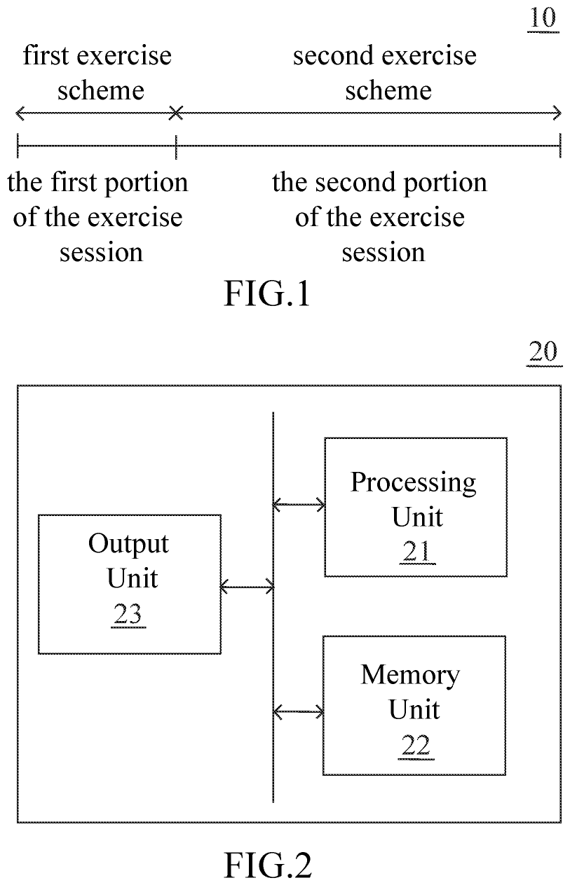

FIG. 1 illustrates a method 10 for executing the multiple schemes to monitor an exercise session of a user in the present invention. For convenience of description, a first exercise scheme and a second exercise scheme are respectively executed in a first portion of the exercise session and a second portion of the exercise session; however, the multiple schemes can be respectively executed in more than two portions of the exercise session. If the real-time fitness condition of the user is inconsistent with the fitness condition in the exercise record of the previous exercise of the user in the first portion of the exercise session, the present invention will suggest a next modified scheme to monitor the second portion of the exercise session. The second portion of the exercise session may follow the first portion of the exercise session. The beginning of the second portion of the exercise session may be the end of the first portion of the exercise session. The beginning of the first portion of the exercise session may be the beginning of the exercise session or there is an interval between the beginning of the first portion of the exercise session and the beginning of the exercise session. The first portion of the exercise session may have a duration of about 0.1-10, 0.1-8, 0.1-6, 0.1-5, 0.1-4, 0.1-3, 0.1-2, 0.1-1, 0.1-0.6 minutes. The duration of the first portion of the exercise session may have 0.0001-10%, 0.0001-8%, 0.0001-6%, 0.0001-5%, 0.0001-4%, 0.0001-3%, 0.0001-2%, 0.0001-1% or 0.0001-0.5% of the duration of the overall exercise session.

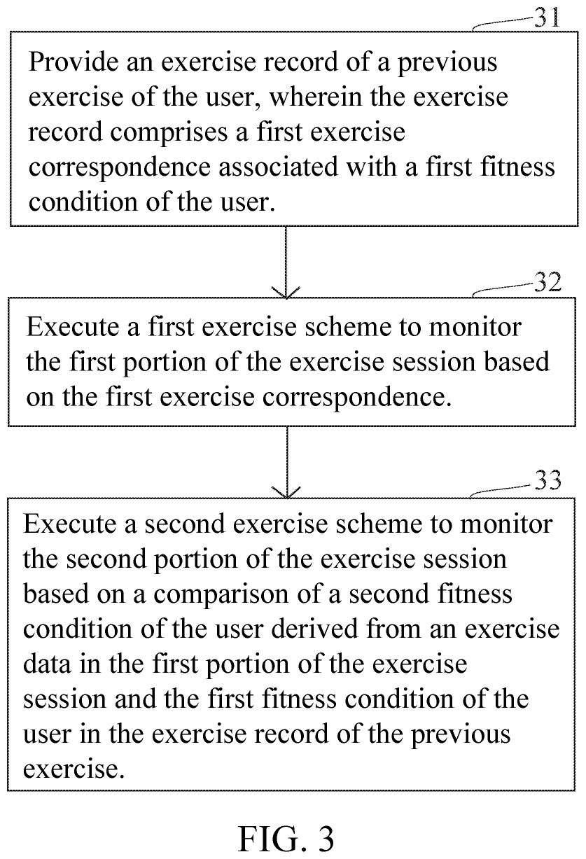

The method in the present invention can be applied in all kinds of apparatuses, such as a wrist top device or any other mobile device. FIG. 2 illustrates a schematic block diagram of an exemplary apparatus 20 in the present invention. The apparatus 20 may comprise a processing unit 21, a memory unit 22 and an outputting unit 23. The apparatus 20 may further comprise an input unit (e.g., a sensor for measuring heart rate), but it is not shown herein. The processing unit 21 may be any suitable processing device for executing software instructions, such as a central processing unit (CPU). The memory unit 22 may include random access memory (RAM) and read only memory (ROM), but it is not limited to this case. The memory unit 22 may include any suitable non-transitory computer readable medium, such as ROM, CD-ROM, DVD-ROM and so on. Also, the non-transitory computer readable medium is a tangible medium. The non-transitory computer readable medium includes a computer program code. The memory unit 22 and the computer program code are configured with the processing unit 21 to cause the apparatus 20 to perform desired operations (e.g., operations listed in claims). The outputting unit 23 may be a display for displaying exercise guiding, exercise scheme or exercise index.

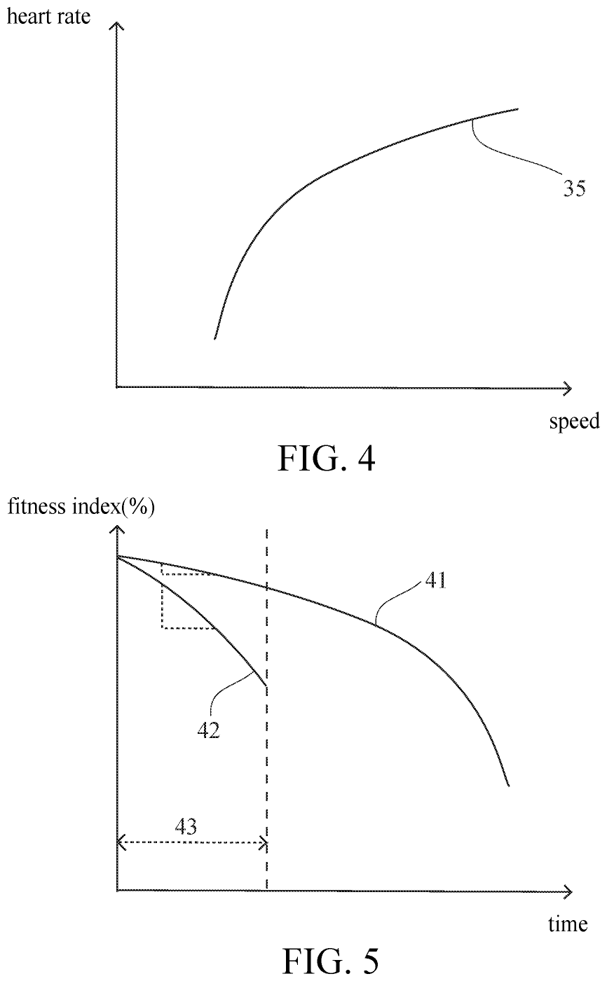

FIG. 3 illustrates a method for monitoring an exercise session of a user in the present invention. The exercise session comprises a first portion and a second portion following the first portion. The method comprises:

Step 31: providing, by a memory unit, an exercise record of a previous exercise of the user, wherein the exercise record comprises a first exercise correspondence associated with a first fitness condition of the user;

Step 32: executing, by a processing unit, a first exercise scheme to monitor the first portion of the exercise session based on the first exercise correspondence; and

Step 33: executing, by the processing unit, a second exercise scheme to monitor the second portion of the exercise session based on a comparison of a second fitness condition of the user derived from an exercise data in the first portion of the exercise session and the first fitness condition of the user in the exercise record of the previous exercise.

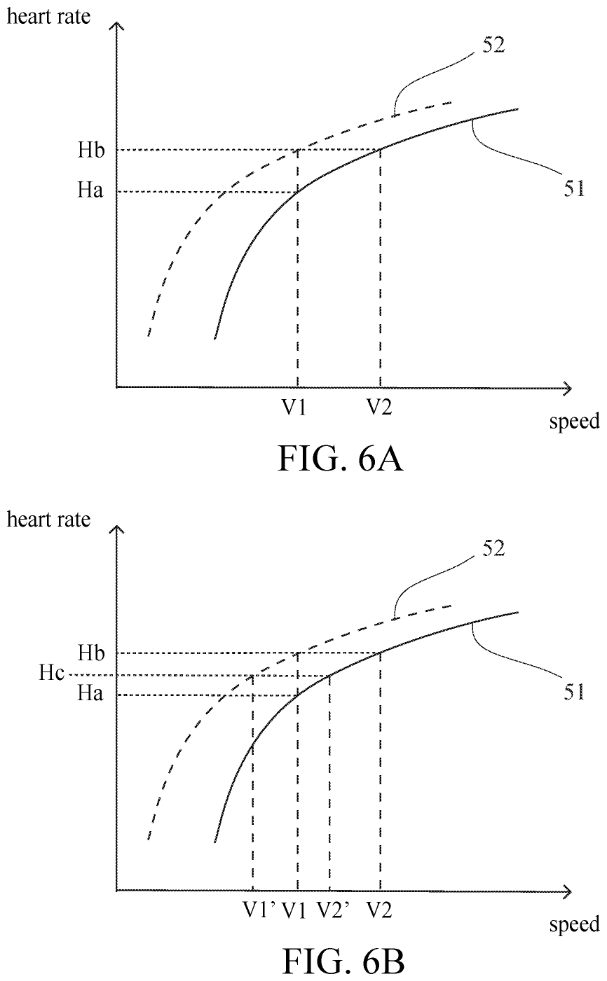

When the user takes exercise in an exercise session, the exercise-monitoring apparatus may suggest an initial exercise scheme for the user in the overall exercise session. For example (I), when the user wants to finish a running session having a target “Training Effect 4”, the exercise-monitoring apparatus may suggest about 8 kilometers per hour (or 7-9 kilometers per hour) in 90 minutes (e.g., first exercise scheme) for the user. The exercise session is monitored based on the first exercise correspondence 35 existing in the exercise record of the previous exercise of the user. The first exercise correspondence 35 is associated with a first fitness condition of the user existing in the exercise record of the previous exercise of the user. The fitness performance level representing the first fitness condition of the user is 36 in example (I). FIG. 4 illustrates a first exercise correspondence 35 associated with the first fitness condition of the user in one embodiment of the present invention. The first fitness condition of the user may be an average fitness condition in the long-term exercise record of the user or a fitness condition in the previous exercise record. The first exercise correspondence 35 may be associated with an exercise intensity. The first exercise correspondence 35 may be associated with a plurality of personalized exercise intensity zones of the user. The first exercise correspondence 35 may describe a dependence of a first parameter on a second parameter. The first parameter may be a parameter associated with the cardiac output power and the second parameter may be a parameter associated with the exercise intensity (of an external workload). The first parameter may be a heart rate, pulse or respiration rate. The second parameter of the exercise intensity may be speed, cycling cadence, force, pedaling power or motion intensity. For convenience of description, the first parameter of the cardiac output power is heart rate and the second parameter of the exercise intensity is speed. Generally, a unit of the heart rate is beats per minute (BPM). The heart rate may be in the form of the percentage of the heart rate reserve (% HRR). The percentage of the heart rate reserve represents the ratio of the difference between the heart rate and the resting heart rate to the difference between the maximum heart rate and the resting heart rate. Generally, a unit of the speed is kilometers per hour.

Please refer the example (I) again, the user has already run for 5 minutes, the second fitness condition of the user in this duration of 5 minutes of the first portion of the exercise session may be different from the first fitness condition in the exercise record of the previous exercise of the user; in other words, it is impossible that the first exercise scheme is executed to monitor the second portion (e.g., remaining portion) of the exercise session based on first exercise correspondence 35; therefore, the present invention proposes a method to monitor the exercise session including (A) deriving the second fitness condition of the user from an exercise data in the first portion of the exercise session; (B) comparing a second fitness condition of the user derived from an exercise data in the first portion of the exercise session and the first fitness condition of the user in the exercise record of the previous exercise; and (C) executing a second exercise scheme to monitor the second portion of the exercise session based on the comparison.

The first portion of the exercise session may have a short duration; in order to effectively derive the second fitness condition of the user from the exercise data in the first portion of the exercise session, immediately choosing a reasonable parameter to represent the fitness condition in the short duration is very important. The parameter may be a gradient of an accumulating physiological index. For example, the parameter is a descending rate of the fitness index. The parameter may be a gradient of a physiological index. For example, the parameter is an ascending rate of the cardiac output power (e.g., heart rate). A combination of the multiple parameters may represent the fitness condition. The combination of the multiple parameters may be in the form of a synthetic index (e.g., weighed average, aX+bY). The multiple parameters may comprise at least two of the descending rate of the fitness index, the ascending rate of the cardiac output power, the fitness index and the cardiac output power.

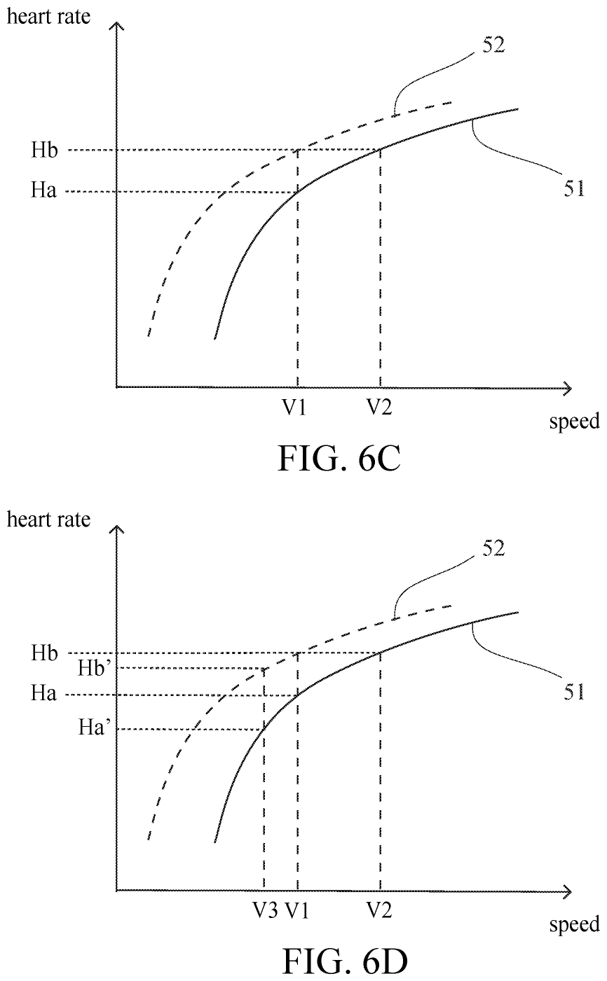

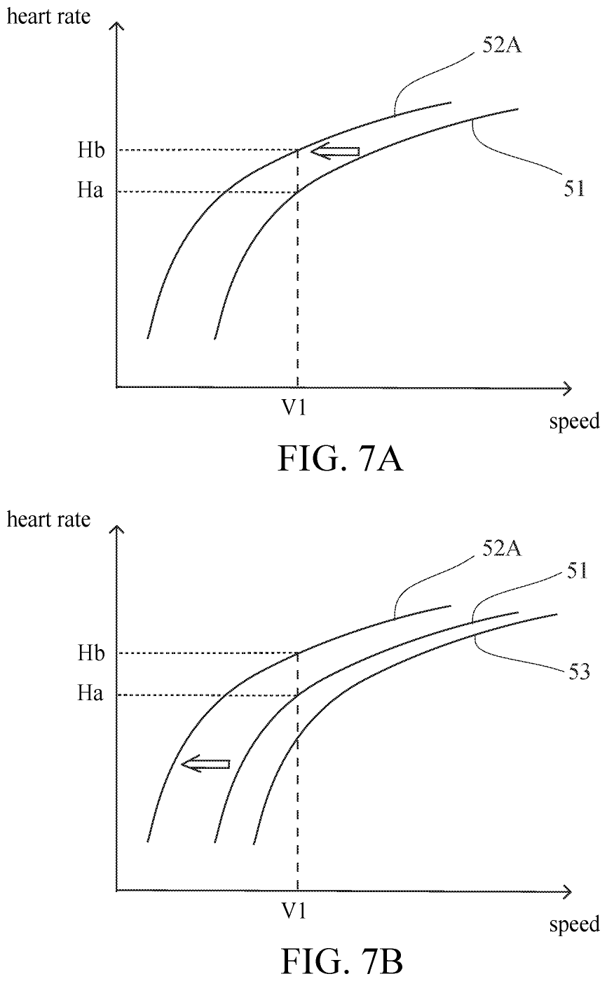

FIG. 5 illustrates that the descending rate of the fitness index is used for representing the second fitness condition of the user in the first portion of the exercise session for subsequent comparison. The descending rate of the fitness index is a better index representing the second fitness condition; however, any suitable parameter can be applied to representing the second fitness condition of the user, such as heart rate shown in subsequent FIG. 6A. For convenience of description, the example (I) is included in FIG. 5 for reference. The curve 41 describes that the fitness index varies with time when the user of the first fitness condition runs at the speed of 8 kilometers per hour in the exercise record of the previous exercise. The curve 42 describes that the fitness index varies with time when the user of the second fitness condition runs at the speed of 8 kilometers per hour in the first portion of the exercise session. The descending rate of the fitness index in the curve 42 is compared to the descending rate of the fitness index in the beginning portion of the curve 41, wherein the curve 42 has a duration 43 of the first portion of the exercise session and the beginning portion of the curve 41 has a duration corresponding to the duration 43 of the first portion of the exercise session.

FIG. 5 specifically shows that the descending rate of the fitness index 2.5% per minute in the curve 42 is much larger than that 1% per minute in the beginning portion of the curve 41 when the user runs at the speed of 8 kilometers per hour. Because the descending rate of the fitness index varies with the fitness condition, so it is reasonable to conclude that the real-time fitness condition in this exercise is much lower than that in the exercise record of the previous exercise.

The difference of the descending rate of the fitness index in the beginning portion of the curve 41 and the descending rate of the fitness index in the curve 42 is used for the comparison of the fitness condition. The difference of the descending rate of the fitness index may be compared to a threshold the user defines; if the difference of the descending rate of the fitness index is less than the threshold, execute the second exercise scheme the same as the first exercise scheme to monitor the second portion of the exercise session; if the difference of the descending rate of the fitness index is larger than the threshold, execute the second exercise scheme different from the first exercise scheme to monitor the second portion of the exercise session. Please refer to the example (I) again, the threshold is 0.2% per minute and the difference is 1.5% per minute, and then execute the second exercise scheme different from the first exercise scheme to monitor the second portion of the exercise session. The descending rate of the fitness index 2.5% per minute in the first portion of the exercise session may be changed to the corresponding second fitness condition (fitness performance level=18) in the first portion of the exercise session. Please refer to the example (I) again, the threshold is 2 and the difference is 18, and then execute the second exercise scheme different from the first exercise scheme to monitor the second portion of the exercise session. If a combination of the multiple parameters may represent the fitness condition, all of the multiple parameters can be designed to form a synthetic index for the comparison of the fitness condition; however, it is not limited to this case.

The present invention chooses a reasonable parameter (or a combination of the multiple parameters) to precisely represent the fitness condition; acquire a first value of the parameter representing the first fitness condition of the user in the condition A1 (e.g., the exercise record of the previous exercise) and acquire a second value of the parameter representing the second fitness condition of the user in the condition A2 (e.g., real-time exercise session); compare the first value of the parameter and a second value of the parameter to precisely and quickly evaluate the difference of the first fitness condition in the condition A1 and the second fitness condition in the condition A2. Therefore, the present invention further precisely provides a modified exercise scheme in the remaining portion of the real-time exercise session.

There are many ways to execute the second exercise scheme to monitor the second portion of the exercise session.

### Embodiment 1A

An undetermined second exercise correspondence is associated with the second fitness condition of the user derived from the exercise data in the first portion of the exercise session. Each of the first exercise correspondence of the undetermined second exercise correspondence describes a dependence of a first parameter on a second parameter. Execute the second exercise scheme to monitor the second portion of the exercise session based on a first value of the second parameter and the first exercise correspondence associated with the first value of the second parameter. A second value of the second parameter is associated with the undetermined second exercise correspondence in the second portion of the exercise session. One of the first value of the second parameter and the second value of the second parameter is modified to the other. The modified value of the second parameter is adjusted based on a difference of the second parameter associated with the first exercise correspondence and the second parameter associated with the undetermined second exercise correspondence under the condition of the same first parameter.

FIGS. 6A and 6B illustrate that execute the second exercise scheme to monitor the second portion of the exercise session in Embodiment 1A of the present invention. Although the second exercise correspondence 52 associated with the second fitness condition of the user can be derived from the exercise data in the first portion of the exercise session, the second exercise scheme is executed to monitor the second portion of the exercise session based on the first exercise correspondence 51 without determining the second exercise correspondence 52 in Embodiment 1A. The first parameter may be a parameter associated with the cardiac output power and the second parameter may be a parameter associated with the exercise intensity (of an external workload). For convenience of description, the first parameter of the cardiac output power is heart rate and the second parameter of the exercise intensity is speed. Each of the first exercise correspondence 51 of the undetermined second exercise correspondence 52 describes a dependence of the heart rate on the speed. Again, the example (I) is included in FIG. 6A for reference. The first exercise scheme is executed to monitor the first portion of the exercise session based on the first exercise correspondence 51 when the first exercise scheme suggests that the user runs at the speed V1 (e.g., 8 kilometers per hour in example (I)) or the user may want to run at the speed V1 (the speed V1 may be defined by the user). The exercise record of the previous exercise shows that the heart rate of the user is Ha when the user of the first fitness condition runs at the speed V1. Because the user has a real-time second fitness condition in the first portion of the exercise session lower than the first fitness condition in the exercise record of the previous exercise, his heart rate is Hb which may be larger than Ha to run at the speed V1. Therefore, it is impossible that execute the second exercise scheme to monitor the second portion of the exercise session at the speed V1 suggested in the first exercise scheme (or defined by user) only based on the first exercise correspondence 51 and the heart rate Ha because the difference of the heart rate (Hb−Ha) is not within a range. The present invention proposes a method to execute the second exercise scheme to precisely monitor the second portion of the exercise session based on the first exercise correspondence 51 without determining the second exercise correspondence 52, which can save the calculation resource of the computer (e.g., save memory usage). The method includes: the second exercise scheme is executed to monitor the second portion of the exercise session based on the speed V2 and the first exercise correspondence 51 associated with the speed V2; the speed V1 is associated with the undetermined second exercise correspondence 52 in the second portion of the exercise session; one of the speed V2 and the speed V1 is modified to the other; the modified speed V2 or the modified speed V1 is adjusted based on the difference of the speed associated with the first exercise correspondence 51 and the speed associated with the undetermined second exercise correspondence 52 under the condition of the same heart rate Hb. In other words, the user of the second fitness condition still runs at the speed V1 suggested in the first exercise scheme (or defined by himself) in the second portion of exercise session and with the heart rate Hb, but the exercise-monitoring apparatus uses the false speed V2 to monitor the second portion of the exercise session by the first exercise correspondence 51 associated with the false speed V2 and the heart rate Hb.

Please refer to FIG. 6B. Because of the decrease of the fitness condition of the user, the second exercise scheme may suggest that the user runs at the speed V1′ (e.g., 7 kilometers per hour in example (I)) less than V1 and with the reduced heart rate Hc, or the user may want to run at the speed V1′ less than V1 (e.g., the speed V1′ may be defined by the user) and with the reduced heart rate Hc. The exercise-monitoring apparatus uses the false speed V2′ to monitor the second portion of the exercise session by the first exercise correspondence 51 associated with the false speed V2′ and the heart rate Hc. The modified speed V2′ or the modified speed V1′ may be obtained by the relationship V2−V1=V2′−V1′ or V2/V1=V2′/V1′; however, the present invention is not limited to this case.

One of the speed V2′ and the speed V1′ is modified to the other. Because the modified speed V2′ can be associated with the first exercise correspondence 51 as much as possible if the speed V1′ associated with the undetermined second exercise correspondence 52 is known, the degree of association between the modified speed V2′ and the first exercise correspondence 51 increases to further precisely monitor the second portion of the exercise session based on the first exercise correspondence 51 and the modified speed V2′ without determining the second exercise correspondence 52. Because the modified speed V1′ can be associated with the second exercise correspondence 52 as much as possible if the speed V2′ associated with the first exercise correspondence 51 is known, the degree of association between the modified speed V1′ and the undetermined second exercise correspondence 52 increases to further precisely monitor the second portion of the exercise session based on the first exercise correspondence 51 and the speed V2′ without determining the second exercise correspondence 52.

In one embodiment, if the second exercise correspondence 52 further comprises a data of the speed V1″ and the heart rate Hd and the first exercise correspondence 51 further comprises a data of the speed V2″ and the heart rate Hd (not shown), the modified speed V2″ or the modified speed V1″ may be obtained by the relationship V2″/V1″=V2/V1=V2′/V1′; however, the present invention is not limited to this case.

### Embodiment 1B

An undetermined second exercise correspondence is associated with the second fitness condition of the user derived from the exercise data in the first portion of the exercise session. Each of the first exercise correspondence of the undetermined second exercise correspondence describes a dependence of a first parameter on a second parameter. Execute the second exercise scheme to monitor the second portion of the exercise session based on a first value of the first parameter and the first exercise correspondence associated with the first value of the first parameter. A second value of the first parameter is associated with the undetermined second exercise correspondence in the second portion of the exercise session. One of the first value of the first parameter and the second value of the first parameter is modified to the other. The modified value of the first parameter is adjusted based on a difference of the first parameter associated with the first exercise correspondence and the first parameter associated with the undetermined second exercise correspondence under the condition of the same second parameter.

FIGS. 6C and 6D illustrate that execute the second exercise scheme to monitor the second portion of the exercise session in Embodiment 1B of the present invention. Although the second exercise correspondence 52 associated with the second fitness condition of the user can be derived from the exercise data in the first portion of the exercise session, the second exercise scheme is executed to monitor the second portion of the exercise session based on the first exercise correspondence 51 without determining the second exercise correspondence 52 in Embodiment 1B. The first parameter may be a parameter associated with the cardiac output power and the second parameter may be a parameter associated with the exercise intensity (of an external workload). For convenience of description, the first parameter of the cardiac output power is heart rate and the second parameter of the exercise intensity is speed. Each of the first exercise correspondence 51 of the undetermined second exercise correspondence 52 describes a dependence of the heart rate on the speed. Again, the example (I) is included in FIG. 6C for reference. The first exercise scheme is executed to monitor the first portion of the exercise session based on the first exercise correspondence 51 when the first exercise scheme suggests that the user runs with the heart rate Hb or the user may want to run with the heart rate Hb (the heart rate Hb may be defined by the user). The exercise record of the previous exercise shows that the speed of the user is V2 when the user of the first fitness condition runs with the heart rate Hb. Because the user has a real-time second fitness condition in the first portion of the exercise session lower than the first fitness condition in the exercise record of the previous exercise, his speed is V1 which may be lower than V2 to run with the heart rate Hb. Therefore, it is impossible that execute the second exercise scheme to monitor the second portion of the exercise session with the heart rate Hb suggested in the first exercise scheme (or defined by user) only based on the first exercise correspondence 51 and the speed V2 because the difference of the speed (V2−V1) is not within a range. The present invention proposes a method to execute the second exercise scheme to precisely monitor the second portion of the exercise session based on the first exercise correspondence 51 without determining the second exercise correspondence 52, which can save the calculation resource of the computer (e.g., save memory usage). The method includes: the second exercise scheme is executed to monitor the second portion of the exercise session based on the heart rate Ha and the first exercise correspondence 51 associated with the heart rate Ha; the heart rate Hb is associated with the undetermined second exercise correspondence 52 in the second portion of the exercise session; one of the heart rate Ha and the heart rate Hb is modified to the other; the modified heart rate Ha or the modified heart rate Hb is adjusted based on the difference of the heart rate associated with the first exercise correspondence 51 and the heart rate associated with the undetermined second exercise correspondence 52 under the condition of the same speedV1. In other words, the user of the second fitness condition still runs with the heart rate Hb suggested in the first exercise scheme (or defined by himself) in the second portion of exercise session and at the speed V1, but the exercise-monitoring apparatus uses the false heart rate Ha to monitor the second portion of the exercise session by the first exercise correspondence 51 associated with the false heart rate Ha and the speed V1.

Please refer to FIG. 6D. Because of the decrease of the fitness condition of the user, the second exercise scheme may suggest that the user runs with the heart rate Hb′ less than Hb and at the reduced speed V3, or the user may want to run with the heart rate Hb′ less than Hb (e.g., the heart rate Hb′ may be defined by the user) and at the reduced speed V3. The exercise-monitoring apparatus uses the false heart rate Ha′ to monitor the second portion of the exercise session by the first exercise correspondence 51 associated with the false heart rate Ha′ and the speed V3. The modified heart rate Ha′ or the modified heart rate Hb′ may be obtained by the relationship Hb−Ha=Hb′−Ha′ or Hb/Ha=Hb′/Ha′; however, the present invention is not limited to this case.

One of the heart rate Ha′ and the heart rate Hb′ is modified to the other. Because the modified heart rate Ha′ can be associated with the first exercise correspondence 51 as much as possible if the heart rate Hb′ associated with the undetermined second exercise correspondence 52 is known, the degree of association between the modified heart rate Ha′ and the first exercise correspondence 51 increases to further precisely monitor the second portion of the exercise session based on the first exercise correspondence 51 and the modified heart rate Ha′ without determining the second exercise correspondence 52. Because the modified heart rate Hb′ can be associated with the second exercise correspondence 52 as much as possible if the heart rate Ha′ associated with the first exercise correspondence 51 is known, the degree of association between the modified heart rate Hb′ and the undetermined second exercise correspondence 52 increases to further precisely monitor the second portion of the exercise session based on the first exercise correspondence 51 and the heart rate Ha′ without determining the second exercise correspondence 52.

In one embodiment, if the second exercise correspondence 52 further comprises a data of the speed V4 and the heart rate Hb″ and the first exercise correspondence 51 further comprises a data of the speed V4 and the heart rate Ha“, the modified speed Hb” or the modified speed Ha″ may be obtained by the relationship Hb″/Ha″=Hb/Ha=Hb′/Ha′; however, the present invention is not limited to this case.

### Embodiment 2

Execute the second exercise scheme to monitor the second portion of the exercise session based on a second exercise correspondence associated with the second fitness condition of the user, wherein the second exercise correspondence is determined by the comparison of the second fitness condition of the user derived from the exercise data in the first portion of the exercise session and the first fitness condition of the user in the exercise record of the previous exercise. The second exercise correspondence determined in Embodiment 2 can be associated with the second fitness condition as much as possible so as to further precisely monitor the second portion of the exercise session based on the determined second exercise correspondence.

### Embodiment 2A

FIG. 7A illustrates that execute the second exercise scheme to monitor the second portion of the exercise session in Embodiment 2A of the present invention. Again, the example (I) is included in FIG. 7A for reference. The first exercise scheme is executed to monitor the first portion of the exercise session based on the first exercise correspondence 51 when the first exercise scheme suggests that the user runs at the speed V1 (e.g., 8 kilometers per hour in example (I)) or the user may want to run at the speed V1 (the speed V1 may be defined by the user). When the user of the first fitness condition runs at the speed V1 in the exercise record of the previous exercise, his heart rate is Ha. Because the user has the real-time second fitness condition lower than the first fitness condition in the exercise session, his heart rate is Hb which may be larger than Ha to run at the speed V1. Therefore, it is impossible that execute the second exercise scheme to monitor the second portion of the exercise session at the speed V1 suggested in the first exercise scheme (or defined by himself) only based on the first exercise correspondence 51 and the heart rate Ha because the difference of the heart rate (Hb−Ha) is not within a range. The present invention proposes a method to execute the second exercise scheme to monitor the second portion of the exercise session based on the second exercise correspondence 52A after determining the second exercise correspondence 52A. The second exercise correspondence 52A may be formed by moving the first exercise correspondence 51 to the point (V1, Hb) along a direction parallel to the axis indicating the speed.

### Embodiment 2B

FIG. 7B illustrates that execute the second exercise scheme to monitor the second portion of the exercise session in Embodiment 2B of the present invention. The exercise record further comprises a third exercise correspondence 53 associated with a third fitness condition of the user, wherein the second exercise correspondence 52A is determined according to a mathematical correspondence constructed from a data set comprising a first data subset and a second data subset, wherein the first data subset comprises the first fitness condition and the first exercise correspondence 51 associated with the first fitness condition, and the second data subset comprises the third fitness condition and the third exercise correspondence 53 associated with the third fitness condition. The second exercise correspondence 52A may be determined according to an extrapolation of the mathematical correspondence of the first exercise correspondence 51 and the third exercise correspondence 53. Again, the example (I) is included in FIG. 7B for reference. The fitness performance level representing the first fitness condition of the user is 36 in example (I). The descending rate of the fitness index is 1% per minute when the user runs at the speed of 8 kilometers per hour. The fitness performance level representing the second fitness condition of the user is 18 in example (I). The descending rate of the fitness index is 2.5% per minute when the user runs at the speed of 8 kilometers per hour. The fitness performance level representing the third fitness condition of the user is 37 in example (I). The descending rate of the fitness index is 0.95% per minute when the user runs at the speed of 8 kilometers per hour. For the same heart rate, the first exercise correspondence 51 has a speed X1, the second exercise correspondence 52A has a speed X and the third exercise correspondence 53 has a speed X2, wherein (X2−X)/(X2−X1)=(0.95−2.5)/(0.95−1). Optionally, for the same speed, the first exercise correspondence 51 has a heart rate Y1, the second exercise correspondence 52A has a heart rate Y and the third exercise correspondence 53 has a heart rate Y2, wherein (Y−Y2)/(Y1−Y2)=(2.5−0.95)/(1−0.95). If the second exercise correspondence 52A is between the first exercise correspondence 51 and the third exercise correspondence 53, the second exercise correspondence 52A may be determined according to an interpolation of the mathematical correspondence of the first exercise correspondence 51 and the third exercise correspondence 53.

### Embodiment 2C

FIG. 7C illustrates that execute the second exercise scheme to monitor the second portion of the exercise session in Embodiment 2C of the present invention. The first exercise scheme may be executed to monitor the first portion of the exercise session based on the first exercise correspondence zone having a left boundary of the first exercise correspondence 51A and a right boundary of the third exercise correspondence 53A. The second exercise correspondence 52A may be determined according to an extrapolation of the mathematical correspondence of the first exercise correspondence 51A and the third exercise correspondence 53A, which is similar to the corresponding description in FIG. 7B and it is not described in detail therein. If the second exercise correspondence 52A is between the first exercise correspondence 51A and the third exercise correspondence 53A, the second exercise correspondence 52A may be determined according to an interpolation of the mathematical correspondence of the first exercise correspondence 51A and the third exercise correspondence 53A.

### Embodiment 3

The exercise record of the previous exercise may further comprise a third exercise correspondence associated with a third fitness condition of the user (not shown). Execute the second exercise scheme to monitor the second portion of the exercise session based on the third exercise correspondence if the tolerance of the second fitness condition and the third fitness condition is within a range, which may be defined by user. Execute the second exercise scheme to monitor the second portion of the exercise session based on the third exercise correspondence if the second fitness condition is substantially consistent with/equal to the third fitness condition.

The above disclosure is related to the detailed technical contents and inventive features thereof. People skilled in the art may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A method for monitoring an exercise session of a user, wherein the exercise session comprises a first portion and a second portion following the first portion, the method comprising steps of:
providing, by a memory unit, an exercise record of a previous exercise of the user, wherein the exercise record comprises a first exercise correspondence associated with a first fitness condition of the user;
executing, by a processing unit, a first exercise scheme to monitor the first portion of the exercise session based on the first exercise correspondence; and
executing, by the processing unit, a second exercise scheme to monitor the second portion of the exercise session based on a comparison of a second fitness condition of the user derived from an exercise data in the first portion of the exercise session and the first fitness condition of the user in the exercise record of the previous exercise.

2. The method according to claim 1, wherein each of at least one parameter representing the first fitness condition and the second fitness condition respectively has a first value and a second value, wherein the comparison of the second fitness condition and the first fitness condition is the comparison of the second value and the first value.

3. The method according to claim 1, wherein the first exercise correspondence is associated with an exercise intensity.

4. The method according to claim 1, wherein the first exercise correspondence describes a dependence of a first parameter on a second parameter.

5. The method according to claim 1, wherein the first exercise correspondence describes a dependence of a cardiac output power on an exercise intensity.

6. The method according to claim 1, wherein an undetermined second exercise correspondence is associated with the second fitness condition of the user derived from the exercise data in the first portion of the exercise session, wherein each of the first exercise correspondence of the undetermined second exercise correspondence describes a dependence of a first parameter on a second parameter,
wherein the second exercise scheme is executed to monitor the second portion of the exercise session based on a first value of the second parameter and the first exercise correspondence associated with the first value of the second parameter, wherein a second value of the second parameter is associated with the undetermined second exercise correspondence in the second portion of the exercise session, wherein one of the first value of the second parameter and the second value of the second parameter is modified to the other.

7. The method according to claim 6, wherein the modified value of the second parameter is adjusted based on a difference of the second parameter associated with the first exercise correspondence and the second parameter associated with the undetermined second exercise correspondence under the condition of the same first parameter.

8. The method according to claim 1, wherein an undetermined second exercise correspondence is associated with the second fitness condition of the user derived from the exercise data in the first portion of the exercise session, wherein each of the first exercise correspondence of the undetermined second exercise correspondence describes a dependence of a cardiac output power on an exercise intensity, wherein the second exercise scheme is executed to monitor the second portion of the exercise session based on a first value of the exercise intensity and the first exercise correspondence associated with the first value of the exercise intensity, wherein a second value of the exercise intensity is associated with the undetermined second exercise correspondence in the second portion of the exercise session, wherein one of the first value of the exercise intensity and the second value of the exercise intensity is modified to the other.

9. The method according to claim 8, wherein the modified value of the exercise intensity is adjusted based on a difference of the exercise intensity associated with the first exercise correspondence and the exercise intensity associated with the undetermined second exercise correspondence under the condition of the same cardiac output power.

10. The method according to claim 1, wherein the second exercise scheme is executed to monitor the second portion of the exercise session based on a second exercise correspondence associated with the second fitness condition of the user, wherein the second exercise correspondence is determined by the comparison of the second fitness condition of the user derived from the exercise data in the first portion of the exercise session and the first fitness condition of the user in the exercise record of the previous exercise.

11. The method according to claim 10, wherein the exercise record of the previous exercise further comprises a third exercise correspondence associated with a third fitness condition of the user, wherein the second exercise scheme is executed to monitor the second portion of the exercise session based on the third exercise correspondence if the tolerance of the second fitness condition and the third fitness condition is within a range.

12. The method according to claim 10, wherein the exercise record further comprises a third exercise correspondence associated with a third fitness condition of the user, wherein the second exercise correspondence is determined according to a mathematical correspondence constructed from a data set comprising a first data subset and a second data subset, wherein the first data subset comprises the first fitness condition and the first exercise correspondence associated with the first fitness condition, and the second data subset comprises the third fitness condition and the third exercise correspondence associated with the third fitness condition.

13. The method according to claim 12, wherein the second exercise correspondence is determined according to an interpolation of the mathematical correspondence.

14. The method according to claim 12, wherein the second exercise correspondence is determined according to an extrapolation of the mathematical correspondence.

15. An apparatus for monitoring an exercise session of a user, wherein the exercise session comprises a first portion and a second portion following the first portion, the apparatus comprising:
a processing unit; and
a memory unit including a computer program code, wherein the memory unit and the computer program code are configured, with the processing unit, to cause the apparatus to perform a process comprising steps of:
providing, by a memory unit, an exercise record of a previous exercise of the user, wherein the exercise record comprises a first exercise correspondence associated with a first fitness condition of the user;
executing, by a processing unit, a first exercise scheme to monitor the first portion of the exercise session based on the first exercise correspondence; and
executing, by the processing unit, a second exercise scheme to monitor the second portion of the exercise session based on a comparison of a second fitness condition of the user derived from an exercise data in the first portion of the exercise session and the first fitness condition of the user in the exercise record of the previous exercise.

16. A method for monitoring an exercise session of a user, wherein the exercise session comprises a first portion and a second portion following the first portion, the method comprising steps of:
providing, by a memory unit, an exercise record of a previous exercise of the user, wherein the exercise record comprises a first exercise correspondence associated with a first fitness condition of the user;
executing, by a processing unit, a first exercise scheme to monitor the first portion of the exercise session based on the first exercise correspondence; and
executing, by the processing unit, a second exercise scheme to monitor the second portion of the exercise session based on a comparison of a second fitness condition of the user derived from an exercise data in the first portion of the exercise session and the first fitness condition of the user in the exercise record of the previous exercise;
wherein a parameter representing the first fitness condition has a first value and the parameter representing the second fitness condition has a second value, wherein the comparison of the second fitness condition and the first fitness condition is the comparison of the second value of the parameter and the first value of the parameter.

17. The method according to claim 16, wherein the parameter is a gradient of an accumulating physiological index.

18. The method according to claim 17, wherein the parameter is a descending rate of a fitness index.

19. The method according to claim 16, wherein the parameter is a gradient of a physiological index.

20. The method according to claim 19, wherein the parameter is an ascending rate of a cardiac output power.
